(19) United States
(12) Patent Application Publication
(10) Pub. No.: US 2024/0212369 A1
(43) Pub. Date: Jun. 27, 2024

(54) MANAGEMENT DEVICE, WEARABLE TERMINAL, AND MANAGEMENT METHOD

(71) Applicant: JVCKENWOOD Corporation, Yokohama-shi (JP)

(72) Inventor: Wataru IKEDA, Yokohama-shi (JP)

(21) Appl. No.: 18/395,967

(22) Filed: Dec. 26, 2023

(30) Foreign Application Priority Data
Dec. 26, 2022 (JP) 2022-208858
Dec. 26, 2022 (JP) 2022-208867

(51) Int. Cl. G06V 20/68; G06V 20/20; G06V 20/40; G06V 20/50; G09B 19/00; G16H 20/60

(52) U.S. Cl. CPC G06V 20/68; G06V 20/20; G06V 20/41; G06V 20/50; G09B 19/0092; G16H 20/60

(57) ABSTRACT

To manage calorie intake per unit time. An AR glasses include: a calorie calculation unit that calculates calories of food or drink ingested by a user per predetermined unit time (for example, 1 minute) based on a video obtained by imaging food or drink consumption by the user; and a management unit that outputs a warning to the user in a case where the calories of the food or drink ingested by the user per unit time calculated by the calorie calculation unit exceed a threshold and determines whether or not the food or drink consumption by the user has been finished, in which the calorie calculation unit and the management unit repeat processes thereof until it is determined that the food or drink consumption has been finished.

### TECHNICAL FIELD

The present invention relates to a management device, a wearable terminal, and a management method.

### BACKGROUND ART

Hitherto, there has been a technique of capturing a still image of a dish or the like using a camera, or the like mounted on a terminal and calculating a calorie intake, a nutritional value, or the like of the dish (for example, PTL 1).

Further, hitherto, there has been a technique of generating advice information regarding a meal based on information such as a meal record transmitted from a user via a network and notifying the user of the advice information in order to perform meal management for example, PTL 2.

### CITATION LIST

### Patent Literature

(PTL 1) JP 2004-118562 A

(PTL 2) JP 2008-59320 A

### SUMMARY OF INVENTION

### Technical Problem

By the way, in general, when the speed of a meal decreases, insulin is appropriately secreted from the pancreas, so that a rise in blood glucose level becomes gradual.

It is possible to obtain the total calories of a meal by using the technique of PTL 1 described above. However, it is not possible to manage the speed of a meal even in a case where the techniques are used.

Further, in general, when protein is ingested prior to carbohydrate and fat, gastrointestinal hormones collectively referred to as incretins are secreted to suppress an increase in blood glucose level. In addition, an increase in blood glucose level is also suppressed by ingesting a food material (for example, mushrooms, vegetables, or the like) rich in water-soluble dietary fiber prior to carbohydrate and fat.

It is possible to provide advice information regarding a meal to a user by using the technique of PTL 2 described above. However, in a case where the technique of PTL 2 is used, only notification of the total calories of a meal and a PFC balance value is made, and it is not possible to manage the order of eating.

The present invention has been made in view of such a problem, and provides a management device, a wearable terminal, and a management method that enable management of a calorie intake per unit time, determination of an appropriate order of eating, and notification to a user.

### Problems to be Solved by the Invention

A management device according to one aspect of the present invention is configured to input video data of a state in which a user is eating or drinking obtained as an imaging result of an imaging unit, acquire information of food or drink ingested by the user based on the input video data, specify an ingestion state of the user based on the acquired information of the food or drink, and output a notification based on the specification result to the user via an output unit.

A management device method according to one aspect of the present invention includes: inputting video data of a state in which a user is eating or drinking obtained as an imaging result of an imaging unit; acquiring information of food or drink ingested by the user based on the input video data: and specifying an ingestion state of the user based on the acquired information of the food or drink and outputting a notification based on the specification result to the user via an output unit.

### Advantageous Effects of Invention

According to the present invention, it is possible to output a notification based on an ingestion state to a user who is eating.

### BRIEF DESCRIPTION OF DRAWINGS

FIG. 1 is a view illustrating an appearance configuration and a use example of augmented reality (AR) glasses 1.

FIG. 2 is a block diagram illustrating an internal configuration of the AR glasses 1.

FIG. 3 is a flowchart describing an operation of the AR glasses 1.

FIG. 4 is a flowchart describing the operation of the AR glasses 1.

FIG. 5 is a diagram describing a change of threshold used for eating in an appropriate order.

FIG. 6 is a diagram describing a change of threshold related to a calorie intake speed.

FIG. 7 is a diagram illustrating an example of information regarding consumption of a current meal.

FIG. 8 is a diagram illustrating another example of the information regarding consumption of a current meal.

### DESCRIPTION OF EMBODIMENTS

### Description of One Embodiment

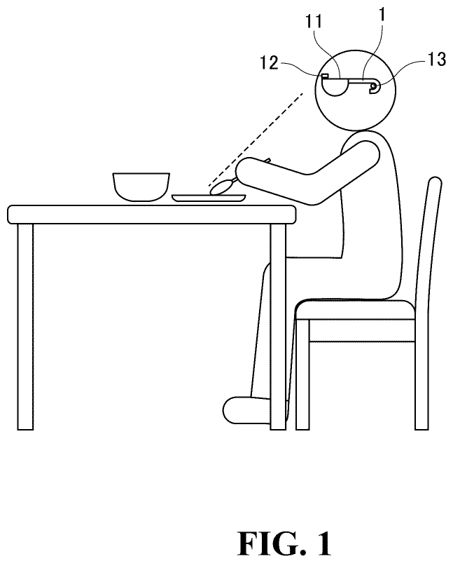

FIG. 1 is a view illustrating an appearance configuration and a use example of augmented reality (AR) glasses 1 according to one embodiment of the present invention.

As illustrated in FIG. 1, the AR glasses 1 are put on both ears to be worn by a user.

The AR glasses 1 are configured in such a way that a pair of display units 11 for the left eye and the right eye is arranged in front of both eyes of the user in a worn state. The display unit 11 displays, for example, a captured image of a real space captured by an imaging unit 12. Furthermore, the display unit 11 may be a transmissive type.

As illustrated in FIG. 1, in the AR glasses 1, the imaging unit 12 is arranged facing forward to perform imaging in a direction visually recognized by the user as a subject direction in a state of being worn by the user.

The AR glasses 1 are provided with an audio output unit 13 including a speaker. The audio output unit 13 is inserted into the right ear hole and the left ear hole of the user in the worn state.

Note that an appearance of the AR glasses 1 illustrated in FIG. 1 is an example, and various structures for the user to wear the AR glasses 1 can be considered.

The AR glasses 1 inputs video data of a state in which the user is eating or drinking obtained as an imaging result of the imaging unit 12, acquires information of food or drink ingested by the user based on the input video data, specifies an ingestion state of the user based on the acquired information of the food or drink, and outputs a notification based on the specification result to the user via the display unit 11 or the audio output unit 13.

For example, the AR glasses 1 image a state in which food or drink as a dish served on a table is conveyed to the mouth of the user, and measure nutrients contained in the food or drink ingested by the user based on the obtained video. Then, the AR glasses 1 determine, based on the measurement result, the order of ingestion of the food or drink in such a way that the ingestion is started from food or drink in which an amount of a predetermined first nutrient is larger than a predetermined threshold, and ingestion is started from food or drink in which an amount of a second nutrient is larger than a predetermined threshold after a predetermined amount of predetermined first nutrient is ingested, and guide the order to the user via the display unit 11 or the audio output unit 13.

Furthermore, for example, the AR glasses 1 image a state in which the food or drink as the dish served on the table is conveyed to the mouth of the user and calculate calories of the food or drink ingested by the user per predetermined unit time that can be measured a plurality of times during a meal based on the obtained video. Then, in a case where the calculated calories of the food or drink ingested by the user per unit time exceeds a threshold, the AR glasses 1 warn the user via the display unit 11 or the audio output unit 13.

### [Internal Configuration of AR Glasses 1]

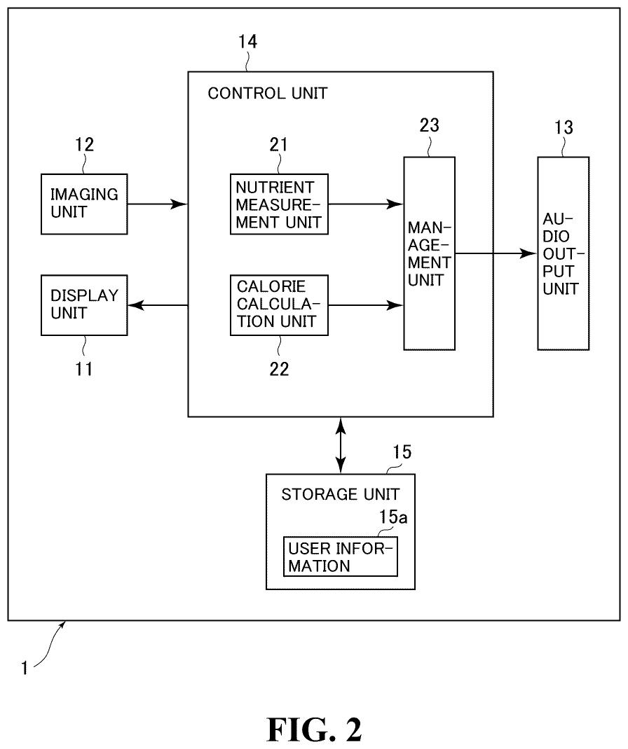

FIG. 2 is a block diagram illustrating an internal configuration of the AR glasses 1. The AR glasses 1 include the display unit 11, the imaging unit 12, the audio output unit 13, a control unit 14, and a storage unit 15.

The display unit 11 displays a video captured by the imaging unit 12, warning information output from the control unit 14, and guidance information for guiding the order of ingestion of the food or drink under the control of the control unit 14. Note that the display unit 11 can display a captured image in real time, and further display the warning information or the guidance information in a superimposed manner in such a way as to correspond to a position of each food or drink in the displayed captured video. In addition, the display unit 11 can display the warning information or the guidance information in such a way as to correspond to the position of the food or drink present in the real space after setting the display unit 11 to a through state (without displaying the captured video).

The imaging unit 12 is, for example, a video camera, and images food or drink consumption by the user, such as a dish for the user or a user's eating or drinking motion, and outputs the imaged video to the control unit 14.

The audio output unit 13 includes, for example, an earphone speaker, and outputs (reproduces) audio signal data under the control of the control unit 14.

Although not illustrated, the control unit 14 serving as a management device includes a central processing unit (CPU), a storage part (a read only memory (ROM), a random access memory (RAM), a nonvolatile memory, or the like), and the like.

The control unit 14 controls the entire AR glasses 1 by executing a control application program (not illustrated) stored in the storage unit 15, and functions as a nutrient measurement unit 21, a calorie calculation unit 22, and a management unit 23.

The nutrient measurement unit 21 measures content of protein (P), fat (F), carbohydrate (C), and dietary fiber content of each served dish and all the dishes based on the video of the food or drink consumption by the user captured by the imaging unit 12.

In addition, the nutrient measurement unit 21 measures nutrients contained in the food or drink ingested by the user based on the video of the food or drink consumption by the user captured by the imaging unit 12. A method for measuring the nutrients may be any method, and for example, image analysis by machine learning or the like can be used.

The calorie calculation unit 22 calculates the calories of each served dish and the total calories of all the dishes based on the video of the food or drink consumption by the user captured by the imaging unit 12.

Furthermore, the calorie calculation unit 22 calculates the calories of the food or drink ingested by the user per predetermined unit time based on the video of the food or drink consumption by the user captured by the imaging unit 12. The calories of the food or drink ingested by the user within a predetermined time are also used as a parameter indicating the speed of the meal. Furthermore, the calorie calculation may be performed by any method, and for example, image analysis by machine learning or the like can be used.

The management unit 23 determines, based on the measurement result of the nutrient measurement unit 21, the order of ingestion of the food or drink in such a way that the ingestion is started from food or drink in which the amount of the predetermined first nutrient is larger than the predetermined threshold, and food or drink in which the amount of the second nutrient is larger than the predetermined threshold is ingested after the predetermined amount of predetermined first nutrient is ingested, and guides the order to the user via the display unit 11 or the audio output unit 13.

In a case where the calories of the food or drink ingested by the user per unit time calculated by the calorie calculation unit 22 exceed the threshold, the management unit 23 warns the user via the display unit 11 or the audio output unit 13.

After the meal is finished, the management unit 23 generates information regarding meal consumption by the user and presents the information via the display unit 11 or the audio output unit 13.

Although not illustrated, the storage unit 15 is composed by a storage part (a ROM, a RAM, a nonvolatile memory, or the like). The storage unit 15 stores the above-described control application program, various data including user information 15a necessary for the execution, and information generated by these processes.

### [Operation of AR Glasses 1]

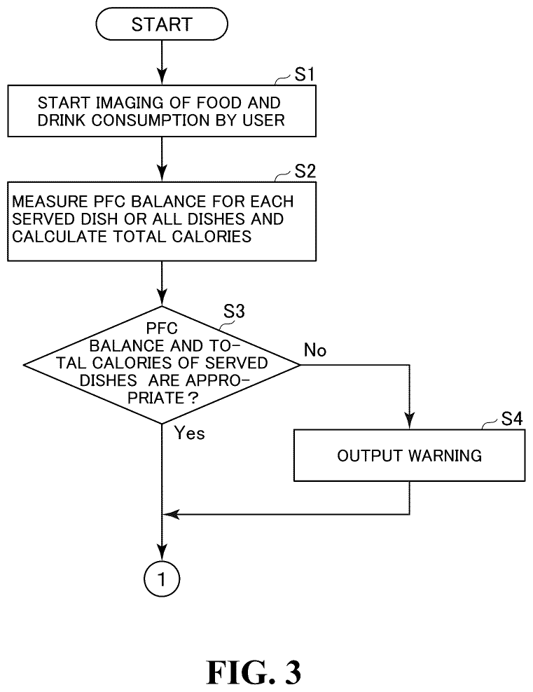

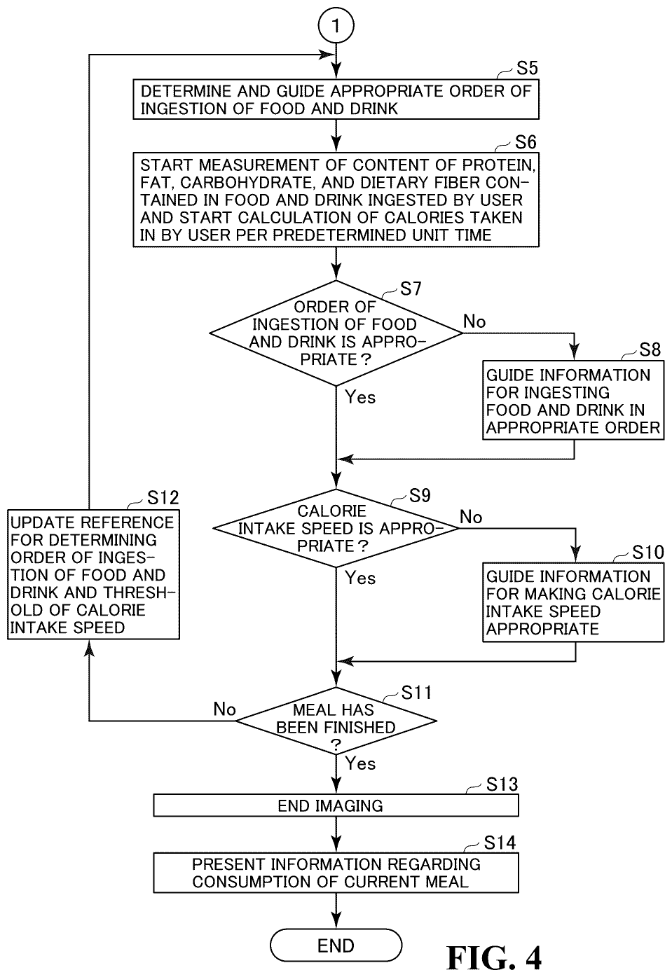

Next, an operation of the AR glasses 1 will be described with reference to the flowcharts of FIGS. 3 and 4. It is assumed that dishes are served on a table, and the user wearing the AR glasses 1 is in a state of ingesting the dishes from now (a state of starting a meal) as illustrated in FIG. 1. In this state, in step S1, the imaging unit 12 starts imaging. The imaging unit 12 images the dish served on the table and also images a state in which food or drink is conveyed to the mouth of the user, that is, the food or drink consumption by the user.

In step S2, the nutrient measurement unit 21 measures the protein (P), fat (F), carbohydrates (C), and dietary fiber content of each served dish and all the dishes based on the video captured by the imaging unit 12. The calorie calculation unit 22 calculates the calories of each served dish or the total calories of all the dishes based on the video captured by the imaging unit 12.

In step S3, the management unit 23 determines whether or not a PFC balance and the calories of the served dish are appropriate based on the measurement result of the nutrient measurement unit 21 and the user information 15a stored in the storage unit 15.

Specifically, for example, the management unit 23 sets an ideal PFC balance to protein (P):fat (F):carbohydrate (C)=1.3 to 2:2 to 2.5:5 to 6, and determines whether or not the PFC balance of all the served dishes measured by the nutrient measurement unit 21 in step S2 falls within the range of the ideal PFC balance.

The management unit 23 also determines whether or not the total calories of all the served dishes calculated by the calorie calculation unit 22 in step S2 exceeds an estimated energy requirement for the user stored as the user information 15a.

The estimated energy requirement is calculated by multiplying a basal metabolic rate (kcal/day) by a physical activity level. The basal metabolic rate (kcal/day) is calculated by multiplying a reference basal metabolic value (kcal/kg/day) by a reference body weight (kg). The physical activity level is calculated by dividing the total energy consumption (kcal/day) by the basal metabolic rate (kcal/day) per day. That is, instead of the estimated energy requirement, these parameters for calculating the estimated energy requirement may be stored as the user information 15a, and the management unit 23 may calculate the estimated energy requirement for the user based on the parameters.

In a case where it is determined in step S3 that the PFC balance and the total calories of all the served dishes are not appropriate, the management unit 23 notifies the user of information for warning via the display unit 11 or the audio output unit 13 in step S4. For example, a message such as “The PFC balance and the calories are not appropriate. It would be better to reduce the fat by ** %.” or “There is no problem with the total calories, but the PFC balance is not appropriate. It would be better to reduce carbohydrate by ** %.” is displayed on the display unit 11 or output by voice from the audio output unit 13.

In a case where it is determined in step S3 that the PFC balance and the total calories of the entire meal are appropriate, or in a case where the user is notified of a warning in step S4, the management unit 23 determines the order of ingestion of the served dishes and guides the order to the user in step S5.

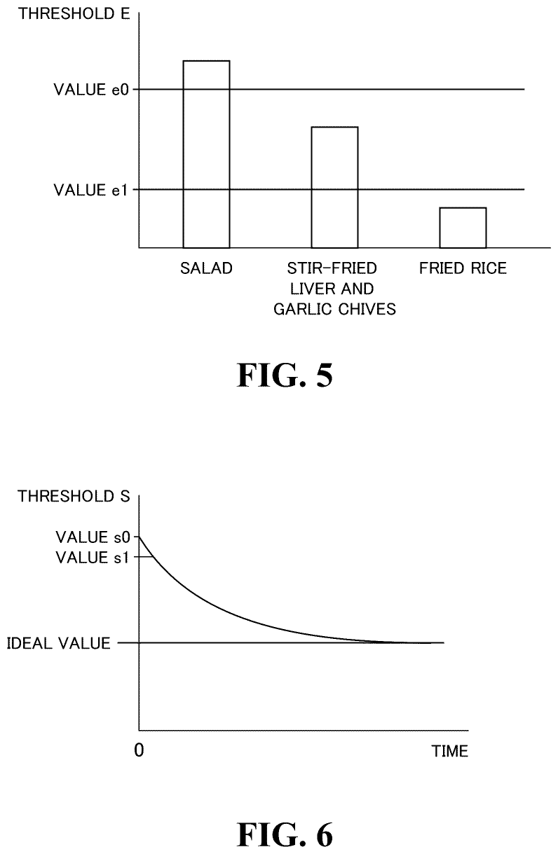

For example, it is assumed that food or drink having a high proportion of protein (P) or food or drink having a large amount of dietary fiber is first ingested. As illustrated in FIG. 5, when first guiding the order of ingestion, that is, at a timing before the user ingests food or drink, the management unit 23 sets a value of a threshold E for the dietary fiber to a value e0, selects a salad in which the amount of the dietary fiber is equal to or more than the threshold E (=value e0) as a dish to be ingested first, and guides the selected salad. In addition to the dietary fiber, the threshold E is provided for each of the protein (P), fat (F), carbohydrate (C), and the dietary fiber.

Then, at a timing when the amount of the food or drink having a high proportion of protein (P) or a large amount of dietary fiber decreases to a predetermined amount (for example, half) or less from the start of eating, food or drink in which a proportion of the fat (F) or carbohydrate (C) is higher than a predetermined threshold is ingested. As described below, the threshold E for each nutrient is updated as the meal progresses in the process of step S12, and the management unit 23 selects a dish to be ingested based on the updated threshold E and guides the dish in the process of the next step S5.

The order of the served dishes determined in this manner is guided to the user.

As a specific example of the guidance to the user, the user is notified of the determined order of the meal by, for example, a text or voice message. Note that, if the determined order of ingestion of the food or drink is not changed, notification of a new text or voice message may be omitted. In addition, it is also possible to present the correct order of ingestion of food or drink by a method that is intuitively easy for the user to understand by performing a process such as displaying a marker or the like superimposed on a part of food or drink that the user may eat, displaying a cross mark or the like superimposed on a part of food or drink that the user should not eat yet on the display unit 11, or performing a mosaic processing on a part of food or drink that the user should not eat yet to make it difficult for the user to see the part.

An initial value of the threshold E may be experimentally and empirically determined or may be appropriately settable by the user.

Next, in step S6, the nutrient measurement unit 21 starts measurement of the content of the protein, fat, carbohydrate, and dietary fiber contained in the food or drink ingested by the user based on the video of the food or drink consumption by the user captured by the imaging unit 12. Specifically, the content of nutrients contained in food or drink are measured from a video of the food or drink conveyed to the mouth by means of chopsticks or a spoon or a video of the food or drink conveyed directly to the mouth from a bowl or cup among videos of the food or drink consumption by the user captured by the imaging unit 12.

Furthermore, the calorie calculation unit 22 starts to calculate calories consumed by the user per predetermined unit time (for example, one minute) that can be measured a plurality of times during a meal every time the user ingests the food or drink based on the video of the food or drink consumption by the user captured by the imaging unit 12. Specifically, the calories of food or drink are calculated from a video of the food or drink conveyed to the mouth from chopsticks or a spoon or a video of the food or drink conveyed directly to the mouth from a bowl or a cup among videos of the food or drink consumption by the user captured by the imaging unit 12.

In step S7, the management unit 23 compares whether or not the PFC balance of the food or drink ingested by the user measured in step S6 corresponds to the PFC balance in a case of the order of ingestion of the food or drink guided in step S5 and determines whether or not the order of ingestion of the food or drink is appropriate.

In a case where it is determined in step S7 that the order of ingestion of the food or drink is not appropriate, the management unit 23 guides information for ingestion of the food or drink in an appropriate order via the display unit 11 or the audio output unit 13 in step S8. For example, the management unit 23 generates data for calling attention with a warning message, a warning sound, or the like, and outputs the data via the display unit 11 or the audio output unit 13. Specifically, it is also possible to present the correct order of a meal by a method that is intuitively easy for the user to understand by performing a process such as displaying a marker or the like superimposed on a part of a dish that the user may eat on the display unit 11, displaying a cross mark or the like superimposed on a part of a dish that the user should not eat yet, or performing a mosaic processing on a part of a dish that the user should not eat yet to make it difficult for the user to see the part.

In a case where it is determined in step S7 that the order of the meal is appropriate, or after information for ingesting the food or drink in an appropriate order is guided in step S8, the management unit 23 determines whether or not the calories (hereinafter, referred to as a calorie intake speed as appropriate) of the food or drink ingested by the user per unit time calculated by the calorie calculation unit 22 in step S6 are higher than a predetermined threshold in step S9.

In general, a calorie intake speed of 30 kcal or less per minute is recommended, but for example, at the beginning of a meal on an empty stomach, it is expected that a meal speed becomes faster and a calorie intake speed becomes faster than the recommended speed. Therefore, as illustrated in FIG. 6, a value of a threshold S of the calorie intake speed (=value s0) is set to be large at the beginning of a meal. Then, as described below, in the process of step S12, the threshold S of the calorie intake speed is changed in such a way as to approach an ideal value as the meal progresses (a value s1 and the like).

In a case where it is determined in step S9 that the calorie intake speed is not appropriate, the management unit 23 guides information for making the calorie intake speed appropriate via the display unit 11 or the audio output unit 13 in step S10. For example, a warning message is displayed on the display unit 11, and a warning sound is output from the audio output unit 13.

In a case where it is determined in step S9 that the calorie intake speed is appropriate, or after the information for making the calorie intake speed appropriate is guided in step S10, the management unit 23 determines whether or not the meal has been finished in step S11. For example, in a case where a video of food or drink is no longer included in the video captured by the imaging unit 12, the management unit 23 determines that the meal has been finished. In a case where the video of the food or drink conveyed to the mouth of the user has not been captured by the imaging unit 12 for a predetermined time or more, the management unit 23 may determine that the meal has been finished.

In a case where it is determined in step S11 that the meal has not been finished, the management unit 23 changes the value of the threshold E also for the protein (P), the fat (F), the carbohydrate (C), and the dietary fiber serving as references for determining the order of ingestion of the food or drink in step S12. For example, in the example of FIG. 5, the value of the threshold E for the dietary fiber is changed from the value e0 to a value e1 (e0>e1).

As illustrated in FIG. 6, the threshold S of the calorie intake speed is changed from the value s0 to the value s1 (s0>s1).

The management unit 23 may update one of the threshold E and the threshold S, or may update both the threshold E and the threshold S. The management unit 23 may update both the threshold E and the threshold S at the same timing or at different timings.

In a case where it is determined that the meal has not been finished, the management unit 23 may immediately update the threshold E and the threshold S or may update the threshold E and the threshold S after a certain period of time. In a case where it is determined that the meal has not been finished, the management unit 23 may update the threshold E and the threshold S once every several times.

The management unit 23 does not have to determine whether or not the meal has been finished. The management unit 23 may update the threshold E and the threshold S when a predetermined time has elapsed. The predetermined time may be one minute, a fixed time, or random. That is, the management unit 23 may update the threshold E and the threshold S based on the lapse of time of the meal.

The management unit 23 may determine whether or not the meal is progressing, and update the threshold E and the threshold S when the meal is progressing. The management unit 23 may calculate the degree of progress of the meal as a progress level and update the threshold E and the threshold S based on the progress level. The management unit 23 may update the threshold E and the threshold S when the progress level exceeds a predetermined value. That is, the management unit 23 may update the threshold E and the threshold S based on the progress level.

The management unit 23 may calculate the progress level based on a ratio between the nutrients of all the dishes measured by the nutrient measurement unit 21 and the nutrients ingested by the user by that time point. The management unit 23 may calculate the progress level based on a ratio between the calories of all the dishes calculated by the calorie calculation unit 22 and the calories taken in by the user by that time point. The management unit 23 may calculate the progress level based on the nutrients of all the dishes measured by the nutrient measurement unit 21 and the nutrients ingested by the user by that time point, and the calories of all the dishes calculated by the calorie calculation unit 22 and the calories taken in by the user by that time point.

Thereafter, the process returns to step S5, and the subsequent processes are performed. That is, the management unit 23 repeats the processes of step S5 to step S12 until it is determined that the meal has been finished. That is, the order of ingestion of the dishes is determined based on the updated thresholds E for the nutrients. Since the threshold E is updated in this manner, in the example of FIG. 5, next, stir-fried liver and garlic chives, in which the content of the dietary fiber is less than the value e0 and equal to or more than the value e1, are guided as the next ingestible dish. In this way, it is possible to notify the user that the user may eat another dish with the lapse of time without causing the user to continue to eat only one dish such as salad. As a result, it is possible to improve the quality of the meal of the user while prioritizing food or drink having a high dietary fiber content.

Furthermore, as described above, the threshold S of the calorie intake speed is also changed in such a way as to approach the ideal value as the meal progresses, so that the quality of the meal of the user can be improved without difficulty. In general, the speed of a comfortable meal is not constant. At the beginning of the meal when hunger is high, there is a desire to eat quickly, but as the meal progresses, it becomes less uncomfortable to eat at a lower speed. For example, in a case where the threshold S is set to the ideal value from the beginning, there is a possibility that a warning is frequently generated for a while from the beginning of the meal, and there is a possibility that a comfortable meal environment for the user is disturbed. However, the threshold change can prevent such a situation.

The initial value of the threshold and a rate of a decrease in threshold may be experimentally and empirically determined or may be appropriately settable by the user. In a case where it is determined in step S11 that the meal has been finished, the imaging unit 12 ends the imaging of the food or drink consumption by the user in step S13.

In step S14, the management unit 23 generates information regarding consumption of the current meal and presents the information via the display unit 11 or the audio output unit 13.

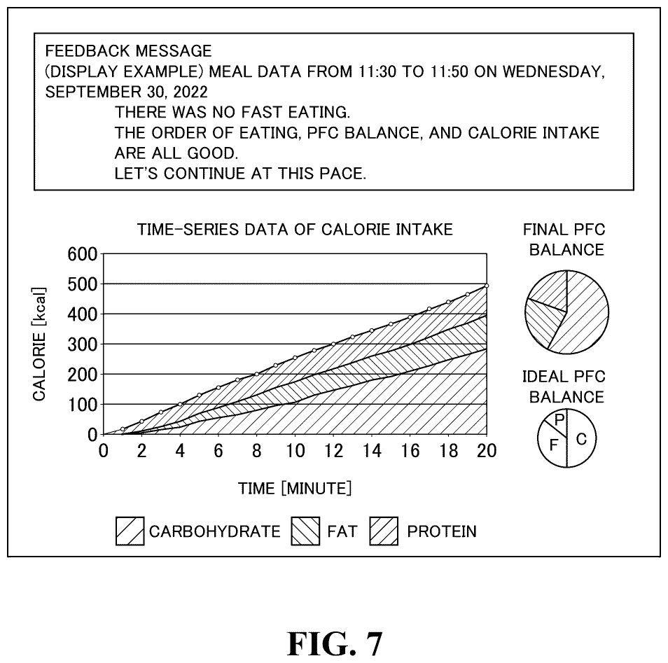

FIG. 7 illustrates an example of the information regarding the consumption of the current meal. The information indicates transition of the PFC balance in the meal, the calorie intake speed in the meal, the PFC balance of the entire current meal, and the ideal PFC balance of the meal together with comments such as whether the consumption of the meal was good or bad.

In the example of FIG. 7, it is illustrated that a larger amount of protein (P) is ingested at the beginning of the meal, and then ingestion of the fat (F) and the carbohydrate (C) gradually increases. The calorie intake speed during the meal is also constant. The PFC balance of the entire current meal is also equivalent to the ideal PFC balance of the meal. In this manner, the user can grasp that the consumption of the current meal was appropriate with reference to FIG. 7.

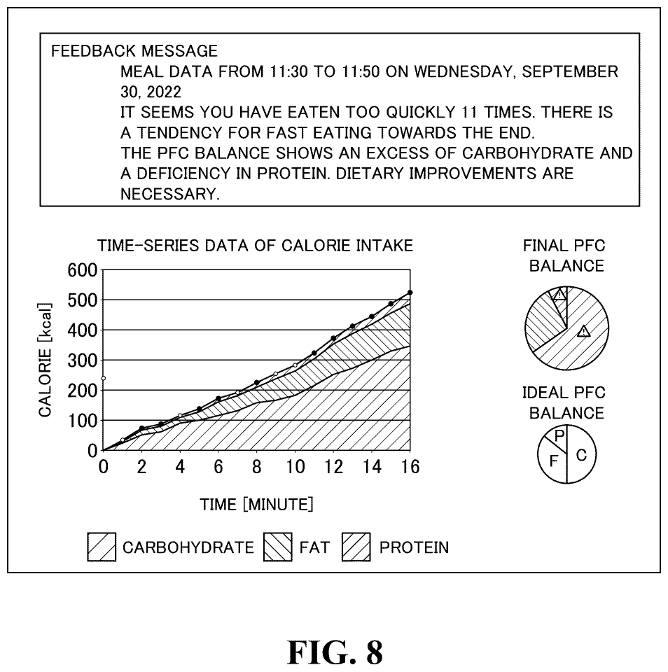

FIG. 8 illustrates another example of the information regarding the consumption of the current meal. In the example of FIG. 8, it is illustrated that a larger amount of carbohydrate (C) is ingested at the beginning of the meal. The calorie intake speed during the meal is also not constant (a slope of the transition of the total calories during the meal is not constant). A portion indicated by a black circle in the graph indicates a fast eating tendency. The PFC balance of the entire current meal also shows a deficiency in protein (P) as compared with the ideal PFC balance of the meal. In this manner, the user can grasp that the consumption of the current meal was inappropriate with reference to FIG. 8.

In step S14, when the information regarding the consumption of the current meal is presented to the user, the process ends.

### Another Example of Information Used for Nutritional Management

In the above example, the description has been given assuming that a moving image of a meal is used as information used for nutrition management, but it is preferable to use other information together for the nutrition management.

For example, in a case where blood glucose level information of the user can be acquired from a blood glucose level sensor, it is possible to notify the user of an increase in blood glucose level 60 to 120 minutes after the end of the meal. In particular, in a case where a high postprandial blood glucose level (140 mg/dL) is confirmed, it is possible to notify the user of a range of the increase in blood glucose level 60 to 120 minutes after the end of the meal, together with problems of the total calories and PFC balance of the meal, and the order and speed of ingestion. In addition, in a case where daily calorie consumption data and an autonomic nerve activity can be acquired from a smart watch, it is possible to determine a physical activity level necessary for deriving the estimated energy requirement for each meal based on the calorie consumption, and it is possible to monitor whether or not the meal does not impose a burden on the gastrointestinal tract based on the autonomic nerve activity.

### Another Example of Calorie Intake Measurement Method

In the above-described embodiment, the AR glasses 1 measure nutrients of food or drink and calculate calories from a video of the food or drink imaged by the imaging unit 12, but a near-infrared nutrition measuring device can also be used. It is also possible to improve calorie intake measurement accuracy by using the above method in combination with the calorie intake measurement method which has been hitherto performed.

### <Information Transmission by Vibration>

In the above-described embodiment, the AR glasses 1 output the information via the display unit 11 and the audio output unit 13. However, the information can be transmitted to the user by vibration, for example. For example, in a case where it is detected that the calorie intake speed is high (step S9), it is also possible to guide appropriate mastication timings by vibration as assistance for making the calorie intake speed appropriate.

### <Other Configurations>

In the AR glasses 1, the display unit 11 may be arranged in front of one eye. That is, in the AR glasses 1, the display unit 11 for the left eye may be arranged, and the display unit for the right eye does not have to be arranged. Alternatively, in the AR glasses 1, the display unit 11 for the right eye may be arranged, and the display unit for the left eye does not have to be arranged.

In the above-described embodiment, a pair of AR glasses 1 include the display unit 11 to the storage unit 15, but these functions may also be implemented by a plurality of devices. For example, the control unit 14 may be separately provided as a management device, be connected to the AR glasses 1 via a network, acquire a video from the imaging unit 12, perform the above-described processes, and display information on the display unit 11 of the AR glasses 1 as appropriate. Some or all of the functions of the display unit 11 to the storage unit 15 can also be implemented by a smartphone. Furthermore, instead of the AR glasses 1, for example, it is also possible to use a wearable terminal capable of acquiring an image of the entire angle of view of the user and superimposing predetermined information on a visual field of the user, such as a head mounted display (HMD).

The management unit 23 may select food or drink containing the first nutrient more than a first threshold based on the measurement result of the nutrient measurement unit 21 and may select food or drink containing the second nutrient more than a second threshold after a predetermined amount of first nutrient is ingested. The management unit 23 may display the selected food or drink on the display unit 11 or output a speech from the audio output unit 13. Furthermore, the management unit 23 may rank the food or drink in descending order of the amount of nutrients and guide the rank to the user.

The management unit 23 may receive an input of the age or gender of the user and store the input as the user information 15a. Furthermore, the management unit 23 may receive an input of a captured image of the user, estimate the age or gender of the user based on the image, and store the estimated age or gender as the user information 15a. Then, the management unit 23 may manage nutrients and calories based on the age or gender of the user. The management unit 23 may change the threshold E and the threshold S based on the age or gender of the user.

In step S3, it is determined whether or not the PFC balance and the total calories of all the served dishes are appropriate. However, it is not always the case that the user ingests all the served dishes. Therefore, the imaging unit 12 may image a state around the user. In addition, the management unit 23 may determine the number of persons who ingest the dishes served on the table, and ages or genders thereof based on the captured video. The management unit 23 may convert the amount of nutrients measured by the nutrient measurement unit 21 or the calories calculated by the calorie calculation unit 22 into an estimated amount expected to be ingested by the user. The management unit 23 may convert the estimated amount by dividing the amount of nutrients or calories by the determined number of persons or may allocate the amount of nutrients or calories based on the ages or genders. Note that, in step S6, the imaging unit 12 images a state in which the food or drink is conveyed to the mouth of the user, and thus, it is not necessary to convert the amount of nutrients or calories into the estimated amount.

In addition, in the flowcharts of FIG. 3 and FIG. 4 described above, the guidance of the order of ingestion of the food or drink in step S5, the determination of whether or not the order of ingestion of the food or drink is appropriate in step S7, the determination of whether or not the calorie intake speed is appropriate in step S9, and the like can be performed without being limited to the order illustrated in FIGS. 3 and 4, and some processes may be omitted.

### Supplementary Description of Embodiments

Each of the above-described embodiments illustrates a preferred specific example of the present invention. Numerical values, constituent elements, arrangement positions of the constituent elements, an order of connection forms, an order of processes in flowcharts, and the like shown in the embodiments are merely examples and are not intended to limit the present invention. Further, each drawing is not necessarily strictly illustrated.

The above-described series of processes can be executed by hardware or software. In a case where the series of processes is executed by software, a program constituting the software is installed from a program recording medium to a computer incorporated in dedicated hardware, or a general-purpose personal computer or the like capable of executing various functions by installing various programs, for example.

Note that the program executed by the computer may be a program in which processes are performed in time series in the order described in the present specification or may be a program in which processes are performed in parallel or at necessary timings such as when a call is made.

### Summary of Effects

(1) The control unit 14 serving as the above-described management device
inputs video data of a state in which the user is eating or drinking obtained as an imaging result of the imaging unit 12,
acquires information of food or drink ingested by the user based on the input video data,
specifies an ingestion state of the user based on the acquired information of the food or drink, and
outputs a notification based on the specification result to the user via the output unit.

With such a configuration, a notification based on the ingestion state of the user can be presented to the user.

(2) The control unit 14 serving as the above-described management device includes:
the calorie calculation unit 22 that calculates calories of food or drink ingested by the user per predetermined unit time (for example, 1 minute) based on a video obtained by imaging food or drink consumption by the user; and
the management unit 23 that outputs a warning to the user in a case where the calories of the food or drink ingested by the user per unit time calculated by the calorie calculation unit exceed a threshold and determines whether or not the food or drink consumption by the user has been finished, in which the calorie calculation unit and the management unit repeat processes thereof until it is determined that the food or drink consumption has been finished.

With such a configuration, it is possible to manage a calorie intake per unit time.

(3) In addition, the management unit 23 decreases the threshold based on the lapse of time of the food or drink consumption.

(4) Furthermore, the management unit 23 calculates the degree of progress of the food or drink consumption as a progress level, and decreases the threshold based on the progress level.

With such a configuration, it is possible to appropriately manage the speed of a meal without disturbing a comfortable meal environment for the user.

(5) Further, the AR glasses 1 include:
the display unit 11 arranged in front of both eyes or one eye of the user in a state where the AR glasses 1 are worn by the user,
the display unit 11 displaying the warning output by the management unit 23 of the control unit 14.

With such a configuration, it is possible to notify the user in an intuitively understandable manner.

(6) The control unit 14 serving as the above-described management device includes:
the nutrient measurement unit 21 that measures nutrients of food or drink ingested by the user based on a video obtained by imaging food or drink consumption by the user; and
the management unit 23 that selects food or drink containing the first nutrient (for example, protein and dietary fiber) more than the first threshold (for example, the threshold E in FIG. 5) based on a measurement result of the nutrient measurement unit 21 and selects food or drink containing the second nutrient (for example, fat and carbohydrate) more than the second threshold after a predetermined amount of first nutrient is ingested.

With such a configuration, it is possible to determine an appropriate order of a meal and notify the user of the order.

(7) Furthermore, the management unit 23 decreases the first threshold (for example, the threshold E in FIG. 5) as the food or drink consumption by the user progresses.

(8) Furthermore, the management unit 23 calculates the degree of progress of the food or drink consumption as a progress level, and decreases the threshold based on the progress level.

With such a configuration, it is possible to determine an appropriate order of a meal and notify the user of the order without disturbing a comfortable meal environment for the user.

(9) Further, the AR glasses 1 include:
the display unit 11 arranged in front of both eyes or one eye of the user in a state where the AR glasses 1 are worn by the user,
the display unit 11 displaying information regarding the order of ingestion of the food or drink determined by the management unit 23.

With such a configuration, it is possible to notify the user in an intuitively understandable manner.

The present disclosure includes matters that contribute to the achievement of the goal aiming “to ensure healthy lives and promote well-being for all at all ages” of sustainable development goals (SDGs) and contribute to value creation by healthcare products/services.

### REFERENCE SIGNS LIST

1 AR glasses
11 Display unit
12 Imaging unit
13 Audio output unit
14 Control unit
15 Storage unit
21 Nutrient measurement unit
22 Calorie calculation unit
23 Management unit

1. A management device configured to:
input video data of a state in which a user is eating or drinking obtained as an imaging result of an imaging unit;
acquire information of food or drink ingested by the user based on the input video data;
specify an ingestion state of the user based on the acquired information of the food or drink; and
output a notification based on the specification result to the user via an output unit.

2. The management device according to claim 1, comprising:
a calorie calculation unit configured to calculate calories of the food or drink ingested by the user per predetermined unit time based on the video data; and
a management unit configured to output a warning to the user in a case where the calories of the food or drink ingested by the user per unit time calculated by the calorie calculation unit exceed a threshold and determine whether or not food or drink consumption by the user has been finished, wherein
the calorie calculation unit and the management unit repeat processes thereof until it is determined that the food or drink consumption by the user has been finished.

3. The management device according to claim 2, wherein
the management unit decreases the threshold based on a lapse of time of the food or drink consumption.

4. The management device according to claim 2, wherein
the management unit calculates a degree of progress of the food or drink consumption by the user as a progress level, and decreases the threshold based on the progress level.

5. A wearable terminal including the management device according to claim 2, the wearable terminal comprising:
a display unit arranged in front of both eyes or one eye of the user in a state where the wearable terminal is worn by the user,
the display unit being controlled by the management unit to display the warning.

6. The management device according to claim 1, wherein:
a nutrient measurement unit configured to measure nutrients of the food or drink ingested by the user based on the video data; and
a management unit configured to select the food or drink containing a first nutrient more than a first threshold based on a measurement result of the nutrient measurement unit and select the food or drink containing a second nutrient more than a second threshold after a predetermined amount of the first nutrient is ingested.

7. The management device according to claim 6, wherein
the management unit decreases the first threshold based on a lapse of time of food or drink consumption.

8. The management device according to claim 6, wherein
the management unit calculates a degree of progress of the food or drink consumption as a progress level, and decreases the threshold based on the progress level.

9. A wearable terminal including the management device according to claim 6, the wearable terminal comprising:
a display unit arranged in front of both eyes or one eye of the user in a state where the wearable terminal is worn by the user,
the display unit being controlled by the management unit to display information regarding an order of ingestion of the food or drink determined by the management unit.

10. A management method executed by a management device that manages a meal, the management method comprising:
inputting video data of a state in which a user is eating or drinking obtained as an imaging result of an imaging unit;
acquiring information of food or drink ingested by the user based on the input video data; and
specifying an ingestion state of the user based on the acquired information of the food or drink and outputting a notification based on the specification result to the user via an output unit.

11. The management method according to claim 10, comprising:
a calorie calculation step of calculating calories of the food or drink ingested by the user per predetermined unit time based on the video data;
an output step of outputting a warning to the user in a case where the calories of the food or drink ingested by the user per unit time calculated by a process of the calorie calculation step exceed a threshold; and
a determination step of determining whether or not food or drink consumption by the user has been finished, wherein
the processes of the calorie calculation step and the output step are repeated until it is determined in the determination step that the food or drink consumption by the user has been finished.

12. The management method according to claim 10, further comprising:
a nutrient measurement step of measuring nutrients of the food or drink ingested by the user based on the video data; and
a selection step of selecting the food or drink containing a first nutrient more than a first threshold based on a measurement result of a process of the nutrient measurement step, and selecting the food or drink containing a second nutrient more than a second threshold after a predetermined amount of the first nutrient is ingested.
